(19) United States
(12) Patent Application Publication
(10) Pub. No.: US 2026/0104426 A1
(43) Pub. Date: Apr. 16, 2026

(54) Proteome Analysis of a Biological Sample by Mass Spectrometry

(71) Applicant: MRM Proteomics Inc., Montreal (CA)

(72) Inventors: Christoph Borchers, Montreal (CA); Rene Zahedi, Winnipeg (CA); Yassene Mohammed, Leiden (NL)

(21) Appl. No.: 19/360,363

(22) Filed: Oct. 16, 2025

Related U.S. Application Data
(60) Provisional application No. 63/707,959, filed on Oct. 16, 2024.

(51) Int. Cl. G01N 33/68; G01N 33/60

(52) U.S. Cl. CPC G01N 33/6848; G01N 33/60; G01N 2333/46; G01N 2458/15

(57) ABSTRACT

Provided herein is a method for protein analysis by mass spectrometry comprising (i) providing one or more internal standards comprising isotope labelled murine proteins obtained from a biological sample from a mouse fed isotopes; and (ii) co-digesting proteins in the biological sample obtained from a same biological sample from the subject, and identifying un-labelled and labelled corresponding doublets of peptides in a mass spectrometry analysis from a lysate mixture from the co-digestion to quantify the proteins from the biological sample from the subject. Further provided are kits for use in the foregoing method. The disclosure further provides methods and kits for the targeted analysis of proteins by designing standards for targeting of the proteins quantified in the foregoing method.

### CROSS-REFERENCE TO RELATED APPLICATIONS

This application claims priority to U.S. provisional patent application No. 63/707,959 filed on Oct. 16, 2024, the contents of which are incorporated herein by reference in their entirety.

### TECHNICAL FIELD

The present disclosure relates to a method for identifying and/or quantifying proteins from a biological sample obtained from a subject and kits for use thereof.

### BACKGROUND

Proteins are the main drivers of disease and comprise the majority of disease biomarkers and drug targets. Mass spectrometry (MS) enables proteome-wide identification and quantitation of proteins and post-translational modifications (PTMs) with high sensitivity, precision, and unmatched specificity in virtually any type of sample, from mummies to human tissue biopsies. Consequently, MS-based proteomics has become the most important tool for biological discovery, with applications ranging from systems biology to biomarker candidate discovery.

Although in recent years throughput and depth of MS-based proteomics approaches have considerably increased while concurrently cost per sample has been reduced, preclinical studies almost exclusively utilize genomics approaches to study larger patient sample cohorts. One major reason why the impact of MS-based proteomics on large-scale studies is clearly lagging behind its unmatched potential to reflect actual disease phenotypes is that most labs exclusively employ relative quantitation to determine fold-changes in protein abundance between samples. Relative quantitation, however, is confined to the comparison of a limited number of samples, that ideally are processed and analyzed together on the same LC-MS system, which otherwise suffers from considerable batch-to-batch effects, which contradict the goals of low-abundance biomarker detection and target identification in heterogeneous patient populations.

Large-scale studies with hundreds to thousands of samples require longitudinal quantitative reproducibility, and eventually data generation across different labs and instruments. To date, these requirements can only be met by absolute quantitation (AbsQuan) where protein concentrations are determined in individual samples using stable isotope labeled (SIL) internal standards. Spiking defined amounts of SIL peptides into samples enables quality control of the entire analytical workflow, including monitoring changes in instrument performance, and thus determining protein concentrations with maximum specificity and precision that can be compared across laboratories and platforms, between studies, and over time. Targeted MS using multiple reaction monitoring (MRM) on triple-quadrupole mass spectrometers is the “gold standard” for absolute protein quantitation, particularly in clinical settings.

The imperative to have SIL peptides for each target protein that are individually synthesized, purified, and characterized (for purity and quantity) drastically increases cost for AbsQuan (ca. $1000 per SIL peptide), confining this powerful technology to a few expert labs that mostly measure small protein panels, even though the feasibility of precise quantitation of hundreds of proteins using highly multiplexed MRM has been demonstrated. As a consequence, MS-based proteomics is clearly dominated by more cost-efficient relative quantitation, which, apart from the abovementioned limitations, also lacks the precision to identify small but biologically relevant changes in protein abundance.

Thus far, there have been challenges with efforts in the field of mass spectrometry to synergize higher-throughput, system-wide discovery with (cost-effective) AbsQuan to provide the unique capacity of precisely determining the concentrations of thousands of proteins across hundreds of samples.

### SUMMARY

The present disclosure seeks to overcome one or more of the foregoing disadvantages or provide useful alternatives thereof.

The disclosure in some embodiments provides high-throughput methods and kits for determining the concentrations of large number of proteins across a plurality of samples (e.g., thousands of proteins across hundreds of samples).

Moreover, in some embodiments, the method of the disclosure may significantly reduce the cost of proteome analysis relative to a widely accepted “gold standard” method for absolute MS-based quantification which requires the synthesis of heavy-labeled internal standards for each protein which are then spiked into each sample.

Such approach, referred to herein as “SysQuan”, repurposes SILAC mouse tissues/biofluids as system-wide internal standards for matched human samples to enable absolute quantitation of, theoretically, two-thirds of the human proteome using 157,086 shared tryptic peptides, of which 73,901 with lysine on the c terminus. In some embodiments, SysQuan may enable quantification of 70% and 31% of the liver and plasma proteomes, respectively.

In further embodiments, the method of the present disclosure may provide one or more of the following advantages for proteome analysis of biological samples: (i) expensive synthetic SIS peptides may be made obsolete; (ii) the ability to provide absolute quantitation of thousands of peptides within one LC-MS run; (iii) the ability to measure several peptides per protein increases the confidence of the assay; (iv) the ability to account for variations in both digestion efficiency (since labelled proteins are added instead of labelled peptides) and matrix effects (lysate of the same organ/biofluid from the labelled mouse is added to the human sample); and (v) both targeted and non-targeted absolute quantitation.

According to one aspect of the disclosure, there is provided an assay kit for a high-throughput proteomics method of identifying and/or quantifying proteins from a biological sample obtained from a subject, the kit comprising (i) one or more internal standards comprising isotope labelled murine proteins obtained from a biological sample from a mouse fed isotopes; and (ii) instructions for co-digesting proteins in the biological sample from a mouse fed isotopes and a corresponding biological sample obtained from the subject, and for identifying unlabelled and labelled corresponding doublets of peptides in a mass spectrometry analysis from a lysate mixture from the co-digestion to quantify the proteins from the biological sample from the subject.

According to another aspect of the disclosure, there is provided a lyophilized internal standard comprising dried isotope labelled murine proteins. In some particularly advantageous embodiments, the internal standard is in powdered form.

In one embodiment, the isotope labelled murine proteins are from a female mouse. In another embodiment, the isotope labelled murine proteins are from a male mouse.

According to another aspect of the disclosure, there is provided a high-throughput method for identifying and/or quantifying proteins from a biological sample obtained from a subject, the method comprising:
(i) providing the biological sample from the subject, wherein the biological sample is from an organ, fluid or tissue;
(ii) homogenizing the biological sample from the subject and extracting proteins therefrom;
(iii) co-digesting proteins in the biological sample obtained from the subject with a corresponding biological sample obtained from a mouse comprising murine proteins, which murine proteins are isotope labelled, the co-digesting producing a lysate mixture comprising a mixture of unlabelled peptides from the biological sample and corresponding isotope labelled murine peptides;
(iv) analyzing the peptides in the lysate mixture by mass spectrometry, wherein the isotope labelled peptides in the murine sample exhibit a measurable mass difference relative to unlabelled peptides from the biological sample from the subject;
(v) identifying unlabelled and labelled corresponding doublets of peptides from the lysate mixture; and
(vi) quantifying the relative abundance of the peptides in the biological sample by comparing an intensity of mass spectrometry signals of the isotope labelled peptides to the unlabelled peptides of the doublets.

In one embodiment, or any one of the foregoing aspects or embodiments thereof, the biological sample obtained from the subject is the fluid.

In one embodiment, or any one of the foregoing aspects or embodiments thereof, the fluid is plasma and wherein the proteins extracted in step (ii) are plasma proteins.

In one embodiment, or any one of the foregoing aspects or embodiments thereof, the biological sample obtained from the subject is from an organ.

In one embodiment, or any one of the foregoing aspects or embodiments thereof, the organ is selected from one of kidney, lung, heart and liver.

In one embodiment, or any one of the foregoing aspects or embodiments thereof, the organ is liver.

In another aspect, there is provided a kit for the foregoing method, the kit comprising a dried isotope labelled murine biological sample. In one embodiment, the dried isotope labelled murine biological sample is lyophilized. In one embodiment, the lyophilized sample is in a powdered form.

According to another aspect, there is provided a kit for quantifying proteins in a sex-specific analysis of the biological sample obtained from a female or male subject, the kit comprising a dried biological sample comprising isotope labelled proteins from one of a respective female mouse or male mouse.

According to another embodiment, the dried biological sample of the mouse comprising isotope labelled proteins is from plasma, kidney, lung, heart, liver.

According to another embodiment, the kit comprises instructions for quantifying the proteins using the method described above.

The kit in any one of the foregoing aspects or embodiments thereof may further comprise software for quantifying the proteins.

According to another aspect there is provided a method for absolute quantification of a protein comprising:
(i) preparing a chemically synthesized unlabeled peptide (NAT) based on a peptide quantified in the method of any one of the above embodiments;
(ii) combining the NAT with one or more isotope-labelled peptides from a mouse to produce a NAT/mouse labelled peptide mixture; and
(iii) analyzing the one or more peptides in the peptide mixture by mass spectrometry, comprising identifying unlabelled and labelled corresponding doublets of the one or more peptides from the peptide mixture, and quantifying the relative abundance of the one or more peptides in the biological sample by comparing an intensity of mass spectrometry signals of the isotope labelled peptides to the unlabelled peptides of the doublets.

In some embodiments, the chemically synthesized peptides are characterized including a determination of the purity and total amount of peptides so that the concentration can be determined for the quantitation of mouse isotopically labeled peptides. These mouse peptides can then be used for absolute quantitation of human peptides.

In one embodiment, the one or more isotope labelled peptides of step (ii) of the foregoing method are stable isotope labelled standard peptides derived from SILAC (SILAC SIS) peptides.

In one embodiment, there is provided an isotope labelled peptide as described in the above method to quantify an endogenous human peptide.

In another aspect, there is provided a kit comprising one or more isotope-labelled peptides of the method described above, the kit optionally further comprising standard operating procedures, reagents, consumables, known protein concentration from a database and/or data analysis software or reference to said software.

The methods, kits, and lyophilized internal standards disclosed herein provide reliable, low-cost high through-put proteomics assays that deliver quantitative analyses of thousands of proteins. The method described herein using SysQuan as a proteome-wide internal standard will allow MS-based large-scale proteomics studies with their unmatched precision and specificity to complement genomics and address the genotype phenotype disconnect.

In some embodiments, the method may allow for the replacement of relative quantitation with affordable absolute quantitation at scale, thus making a given proteomics study and individual sample considerably more informative and valuable for the scientific community. This will be particularly relevant in the study of rare diseases, typically suffering from limited availability of patient samples. The ability to directly compare individual patients and studies, even when single cases have been analyzed and published years earlier, could transform rare disease research. The method may be used as a companion diagnostic and even diagnostic tool, as beyond classical absolute quantitation of biomarkers it will provide direct and clear evidence for the dysregulation of key metabolic pathways—without the need to run patients samples in batches with controls. The ability to compare protein concentrations across different human tissues and different diseases will enable novel study designs that are difficult or impossible with current technology.

### BRIEF DESCRIPTION OF FIGURES

FIG. 1. A schematic of the quantification method using SILAC mouse material to establish relative quantification in human samples.

FIG. 2A. The Venn diagram shows the coverage of the identifiable as well as the quantifiable proteins, in comparison to known plasma proteins according to the Human Plasma Proteome Project (HPPP).

FIG. 2B. A histogram of proteins and how they are covered by heavy-light peptide pairs showing that more than 50% of plasma proteins can be quantified with two or more peptides.

FIG. 3A. Donut plot of the labeling efficiency in the liver with the majority of peptides and proteins showing greater than 90% labeling.

FIG. 3B. Peptide identification shows approximately 20,000 light-heavy peptide pairs of liver.

FIG. 3C. In-depth proteomics is achieved by using high pH pre-fractionation to split the mixed human-SILAC mouse mixed liver sample into 48 fractions with minimal overlap between the fractions.

FIG. 3D. A histogram of proteins and their peptide coverage shows that >50% of liver proteins can be quantified with two or more heavy-light peptide pairs.

FIG. 3E. Agreement in quantification between two sample acquisitions shown in log scale.

FIG. 3F. Comparison of the total number of identifications in the results with the human liver atlas.

FIG. 4A. Exemplary workflow, starting from discovery using processing of matched human and heavy mouse tissue, high pH fractionation and non-targeted data acquisition (DDA), with subsequent data analysis and in-silico targeted assay (MRM) development, and analysis using triple quadrupole (QqQ) MS.

FIG. 4B. Examples of light (top, human) and heavy (bottom, mouse) doublets, identified in targeted MRM acquisition mode.

FIG. 5A. A pie chart showing human proteins with and without shared peptides in mouse.

FIG. 5B. shows overlap of human and mouse tryptic peptides.

FIG. 6. A graphic depicting a method of quantifying proteins according to an embodiment of the disclosure.

FIG. 7A. Generation of SIL mouse reference standards used in the SysQuan strategy.

FIG. 7B. Identification of proteotypic doublets for Absolute Quantification (AbsQuan).

FIG. 7C. Reverse AbsQuan of mouse proteins.

FIG. 7D SysQuan kits and software for AbsQuan of thousands of human proteins.

### DETAILED DESCRIPTION

A detailed description of one or more embodiments of the invention is provided below along with accompanying figures that illustrate the principles of the invention. The invention is described in connection with such embodiments, but the invention is not limited to any particular embodiment described herein. The scope of the invention is limited only by the claims and equivalents thereof. Numerous specific details are set forth in the following description in order to provide a thorough understanding of the invention. It being understood that various changes can be made in the function and arrangement of elements without departing from the scope as set forth in the claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments. These details are provided for the purpose of providing non-limiting examples and the invention may be practiced according to the claims without some or all of these specific details. For the purpose of clarity, certain technical material that is known in the technical fields related to the invention has not been described in detail so that the invention is not unnecessarily obscured by such descriptions.

Articles such as “a” and “an” when used in a claim, are understood to mean one or more of what is claimed or described. It is to be understood that where the specification states that a component feature, structure, or characteristic “may”, “might”, “can” or “could” be included, that particular component, feature, structure, or characteristic is not required to be included.

The term “subject” generally refers to a vertebrate, such as a mammal. The term “mammal” is defined as an individual belonging to the class Mammalia. In some embodiments, the subject is a human. In one embodiment, the subject is a human patient.

The term “SILAC” or “SILAC proteins”, means Stable Isotope Labeling by Amino Acids in Cell Culture. The term includes proteins and peptides isolated from murine samples prepared from mice fed either normal or heavy stable isotope-labeled amino acids, allowing newly synthesized proteins to be labeled metabolically.

“SIL” peptides, as used herein, refers to synthetic isotopically labelled peptides. SIL peptides comprise heavy amino acids derived from natural amino acids by substitution of certain atoms with their heavy isotope variants, such as ¹³C, ¹⁵N and ²H for ¹²C, ¹⁴N and ¹H respectively.

“SIS”, or “stable isotope standard”, refers to one or more synthetically labelled peptides used as internal standards in proteomics analyses to accurately quantify proteins in complex samples.

The term “instructions” refers to one or more written, printed, electronic or graphical materials that describe, direct or illustrate one or more steps for using the component(s) of the kit. The instructions may be provided in physical form (e.g., a printed insert, label or packaging) or in electronic form (e.g., a website link, QR code, downloadable file, software or a display associated with the kit).

Provided herein is a method for the quantitation of proteins (referred to herein as “SysQuan”) for the quantification of proteins in different biological samples (e.g., biofluids, organs, and tissues), potentially at a fraction of the current cost. The method is applicable to human subjects but could be applied to domesticated mammals as well, such as in the agricultural industry or pets. The method described in embodiments herein exploits the genetic proximity between humans and mice, which has made mice the dominant model system in human disease research, with 99% of all human genes having homologs in the mouse genome. While homologous proteins may not have identical amino acid sequences, many sequence stretches that are essential to protein function are identical between mice and humans. In silico digestion with trypsin reveals that approximately 157,000 out of 455,000 human peptides with a length of 7-25 amino acids are also present in mouse, amounting to 68% of all human proteins having shared tryptic peptides with mouse.

This proteomic proximity enables replacing costly individual SIL peptides with system-wide internal standards derived from SIL (stable isotope labelled) tissues that can be extracted in large quantity from SILAC (stable isotope labeling by amino acids in cell culture) mice. Matched “light” human and “heavy” SIL mouse tissues can be pooled and after co-digestion, peptides that are shared between humans and mice will generate light/heavy peptide pairs with defined mass shifts (doublets), just like SIL peptides. The use of SIL mouse tissue stocks as internal standards improves precision when analyzing large sample numbers, as human proteins can be referenced to the same internal standard. However, determining actual protein concentrations in the mouse reference tissues will truly transform quantitative proteomics: Once the concentration of a mouse protein is known in a given reference tissue stock using cost-effective unlabeled “light” peptides, this standard can be used for AbsQuan of this protein in tens of thousands of samples. Concurrently, two major challenges of current protein quantitation may become obsolete: impaired precision and accuracy due to matrix effects and variations in digestion efficiency, both of which cannot be controlled using SIL peptides.

The SysQuan described herein can be used for both targeted proteomics, i.e., MRM and parallel reaction monitoring (PRM), and non-targeted proteomics, i.e., data dependent and data independent acquisition (DDA or DIA). SysQuan will enable retroactive determination of human protein concentrations in untargeted datasets, even if corresponding mouse standard protein concentrations have only been determined years post-acquisition. The inventors believe that SysQuan's unique ability to serve as proteome-wide internal standard will enable MS-based large-scale proteomics studies with the precision and specificity to complement genomics and address the genotype phenotype disconnect.

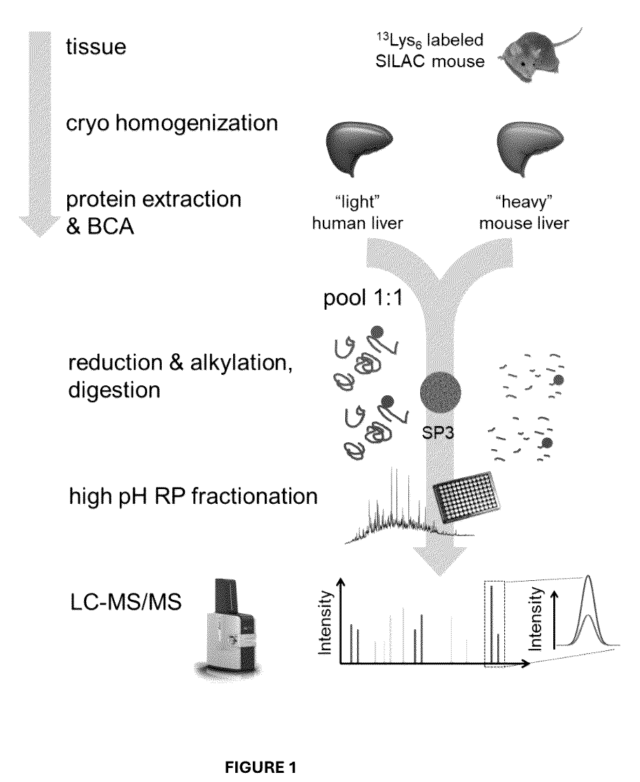

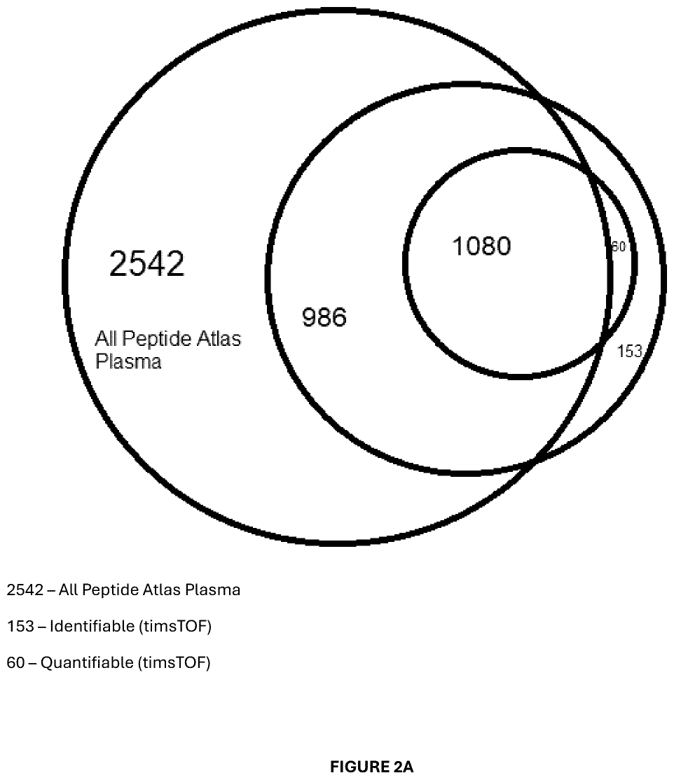

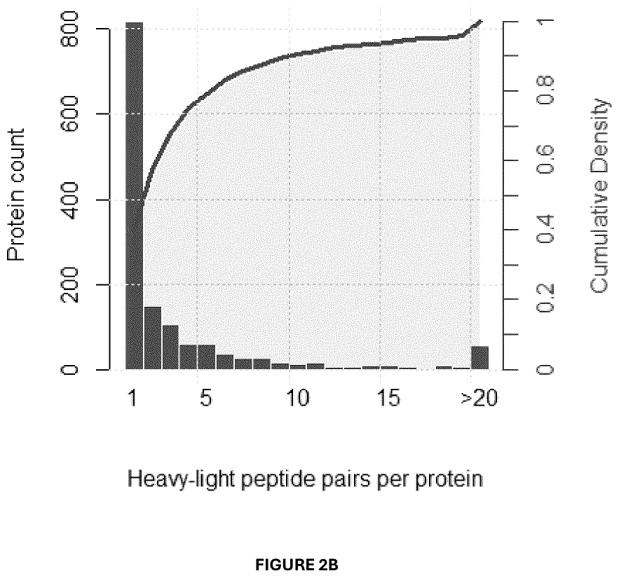

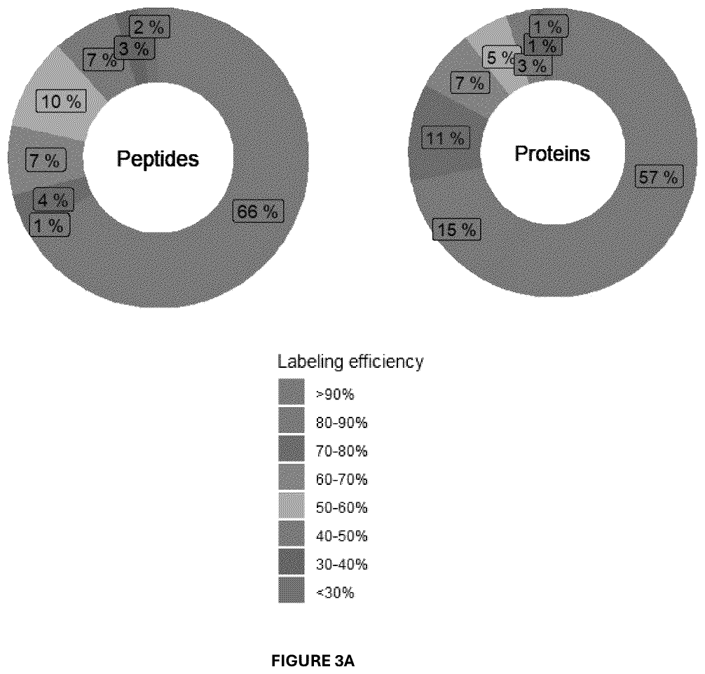

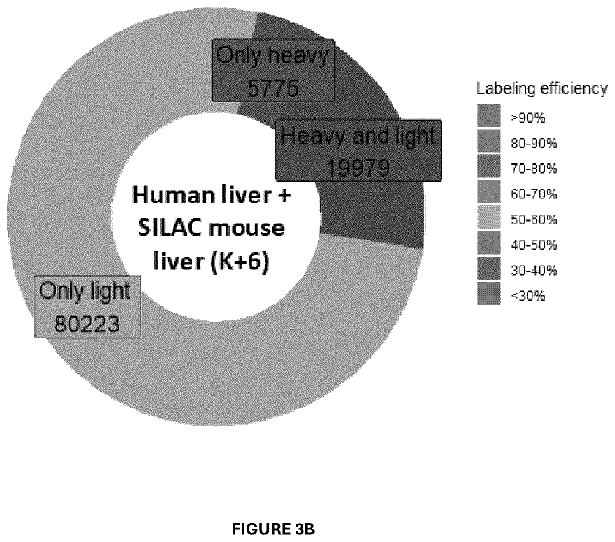

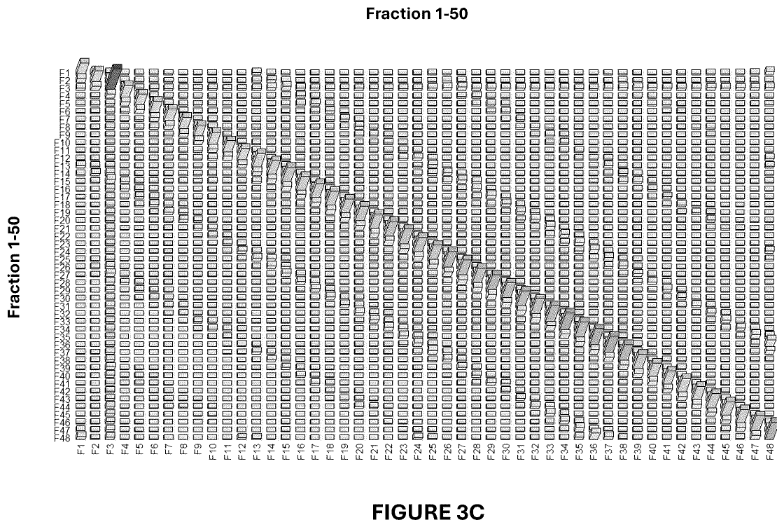

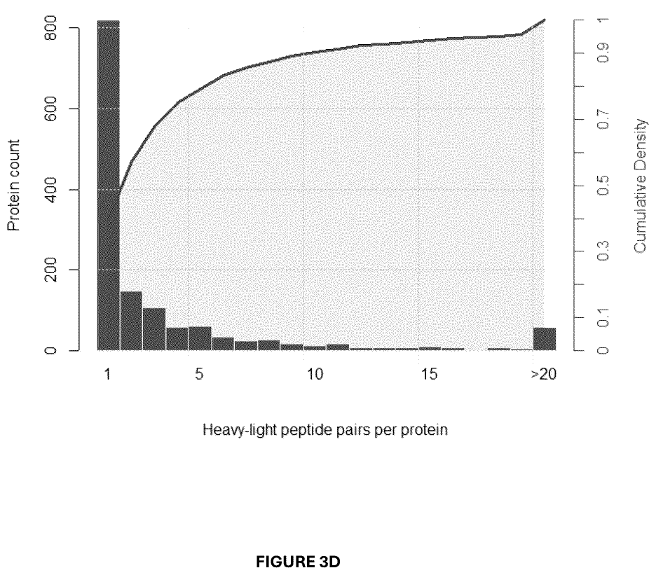

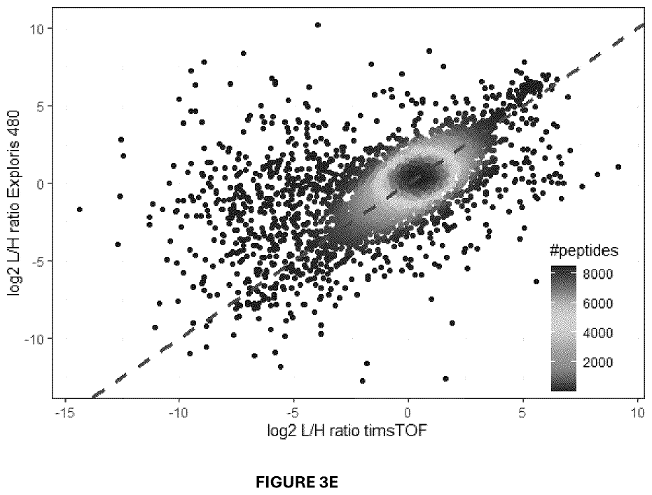

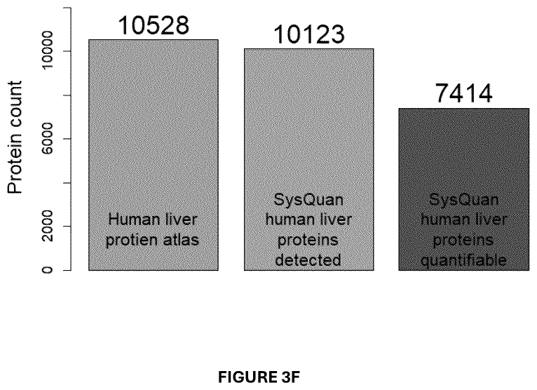

A non-limiting example of one aspect of the inventive method is shown in FIG. 1. A sample, in this case from liver, is obtained from a human and a ¹³Lys⁶ labeled SILAC mouse (described below). Both samples are subjected to cryo homogenization, and then pooled, e.g., at a 1:1 weight ratio. The pooled sample is subjected to reduction and alkylation, followed by proteolytic digestion. After digestion, the pooled sample is subjected to high pH reverse-phase (RP) fractionation to fractionate the peptides based on hydrophobicity and typically at a pH above about 9. MS analysis is carried out and doublets are analyzed. The doublets result from a mass shift between the human light and mouse heavy versions of the peptide. The ratio of the peak intensities in the doublet provides a measure of the amount of the peptide present in the two samples.

SILAC mice result from feeding mice a diet of stable isotope-labelled essential amino acids. Proteins synthesized by the mice incorporate the labelled amino acids, producing a “heavy” version of proteins that can be distinguished by mass spectrometry. The ¹³Lys⁶ labeled SILAC mouse is obtained by feeding a mouse a diet of ¹³Lys⁶ so that the mouse contains proteins with the label. Other isotope-labelled essential amino acids besides ¹³Lys⁶ could be used in the practice of the invention to produce an isotope labeled mouse.

The proteolytic digestion may be carried out with trypsin, although other proteolytic digestion methods known to those of skill in the art are encompassed by the present disclosure (e.g., Lys-C, Asp-N and/or Arg-C digestion). The digestion is most advantageously carried out on the pooled (i.e., combined) protein samples. Mixing of the ‘light’ human and ‘heavy’ mouse tissue prior to proteolytic digestion may eliminate matrix effects and variations in digestion efficiency.

Quantifying proteotypic peptides as surrogates for their corresponding proteins has been demonstrated to be precise. Accuracy requires additional steps, for example optimizing and determining digestion efficiency associated with the target proteotypic peptide release. Standard addition using certified authentic material of known concentration enables accurate quantitation, but such certified materials are, unfortunately, not available for most proteins, and it is often impossible or impractical to obtain them.

Precision is important for preclinical applications, since it encompasses repeatability and reproducibility. For most applications, from discrimination between treatment groups to identification of biomarker candidates, precision is sufficient and traditional targeted proteomics using internal standard provides that required precision. Importantly, data acquired using the inventive approach, with internal standards for hundreds of proteins, can also be reassessed for quantification post-acquisition. Thus, reassessment to achieve a desired accuracy can be performed after information on peptide release and digestion efficiency for a specific protein becomes available. This can be used to achieve absolute quantification later—after SILAC mouse materials have been characterized, which can be performed using unlabelled peptide standards.

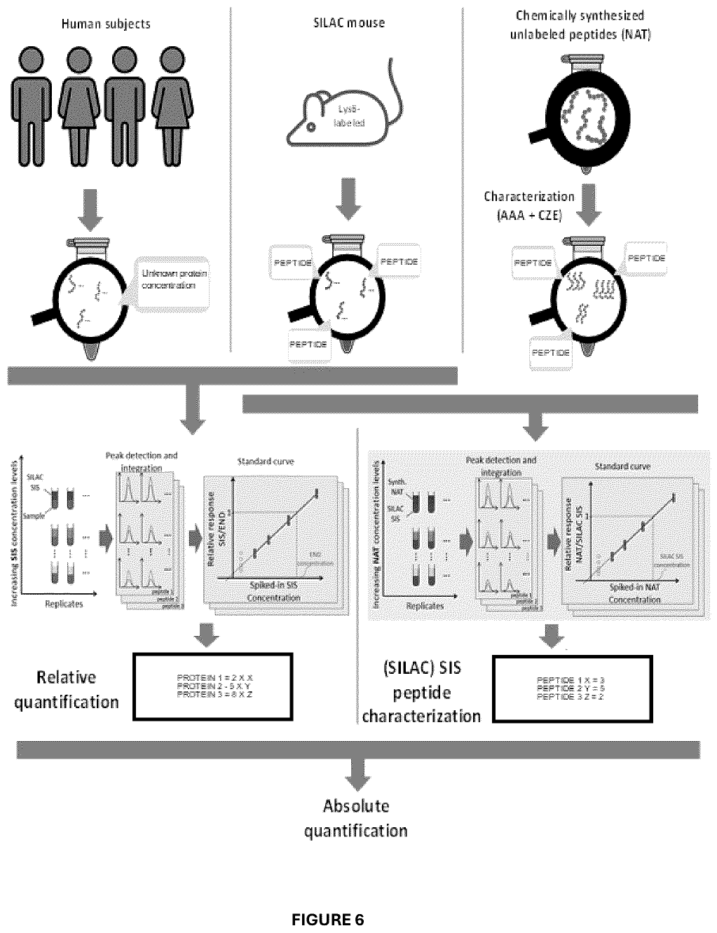

FIG. 6 illustrates an embodiment of the general workflow of such an approach including two analyses: 1) relative quantification as is described above in detail and in the Examples, and 2) characterization of SILAC mouse material using unlabeled synthetic peptides (NAT). The NAT peptides serve as a primary standard to characterize the secondary standards, while the SILAC SIS peptides serve as the secondary standard, i.e., the actual standards used for quantifying the endogenous human peptides. By combining these two steps, absolute quantification of proteins in the target human samples can be achieved.

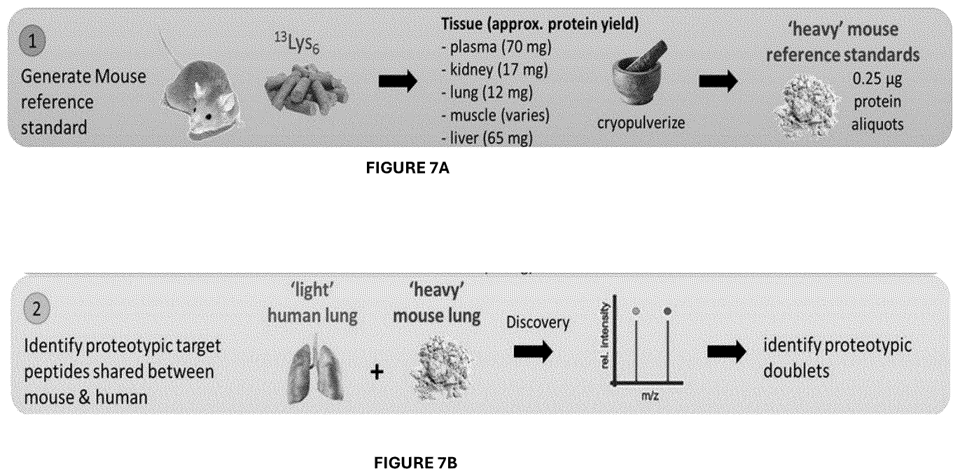

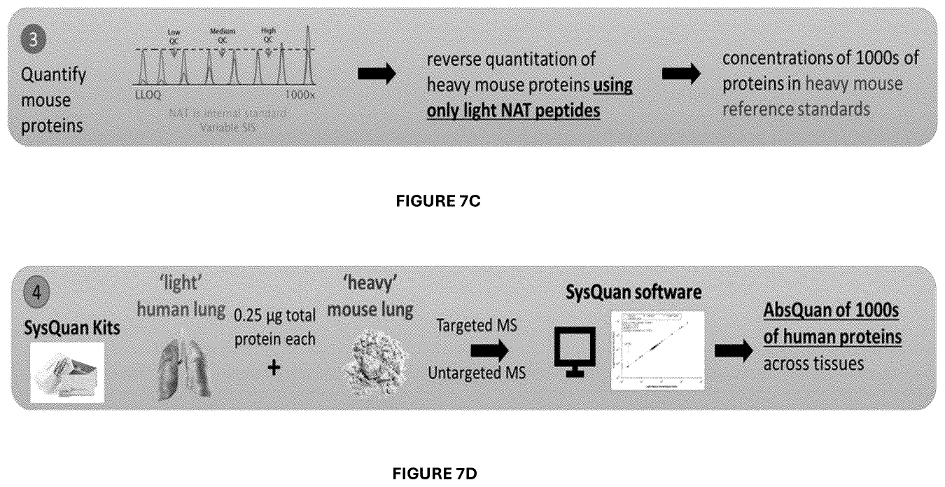

The present disclosure also provides kits. In one embodiment, the SysQuan kit avoids or reduces the need for expensive SIL peptides while expanding AbsQuan towards potentially the entire human proteome (see FIG. 7). In some embodiments, the SysQuan kits will contain lyophilized SIL mouse reference standards for e.g., 100 analyses, known protein concentrations from an updated online database, standard operating protocols (SOPs) and required consumables, and SysQuan software for automated data analysis. In one embodiment, the kit comprises reagents and consumables to prepare samples for mass spectrometry. In another embodiment, the kit comprises all analytical parameters of the LC-MS/MS (liquid chromatography-tandem mass spectrometry) experiments to perform MRM or PRM analysis of the peptide digestion of human and mouse labeled material for absolute quantitation of human proteins.

In one embodiment, the kit comprises isotope-labelled (referred to herein as “heavy”) dried sample from a mouse biological sample. In one embodiment, the kit comprises mouse biological samples of isotope-labelled mouse plasma, kidney, lung, heart or liver.

The following examples describe some exemplary modes of practicing certain methods that are described herein. It should be understood that the examples are for illustrative purposes only and are not meant to limit the scope of the kits and methods described herein.

### EXAMPLES

### Materials and Methods

The following abbreviations are used throughout the Examples:
ACN=acetonitrile
FA=formic acid
RF=radio frequency
SILAC=stable isotope labeling by amino acids in cell culture
SIL=stable isotope-labeled
SIS=stable isotope standard
   Human and SILAC mouse materials

Human plasma was purchased from BioIVT™ (Westbury, NY, USA) and obtained in K₂-EDTA vials and stored at −80° C. until use. Plasma samples were from healthy donors (ages 18 to 50), who provided informed consent. Human fresh-frozen liver samples were obtained from the Jewish General Hospital (JGH), McGill University. Informed consent for the use of the samples in research was provided by the patients. Isotopically labelled mouse plasma and fresh-frozen liver samples were acquired from Silantes™ GmbH (Munich, Germany), product numbers 252923900 and 252923905. The labeling was performed in C57BL6 mice and was confirmed to be 97% on average by Silantes™ GmbH using mass spectrometry.

### Plasma Sample Preparation

Human and mouse plasma samples were depleted of the top 14 most abundant plasma proteins using Thermo Fisher Scientific™ High Select™ Top14 abundant protein depletion columns, in order to reduce the dynamic range of the plasma proteome, thereby maximizing the depth of proteome coverage. Depleted mouse and human plasma samples were pooled 1:1 and subsequently diluted in protein denaturation buffer (5% SDS, 100 mM TRIS pH 8.5, 10 mM TCEP (Tris-(2-carboxyethyl) phosphine)) and heated to 60° C. for 30 minutes. Proteins were reduced in 10 mM TCEP, alkylated with 20 mM iodoacetamide (IAA), and digested with trypsin (Sigma-Aldrich™) at a 1:10 enzyme: substrate ratio (approximately 5 μg of trypsin to 50 μg of depleted plasma protein) at 37° C. for 16 hours using S-TRAP micro cartridges (ProtiFi™ LLC). Proteolysis was stopped with formic acid (FA; 0.5% v/v) and the resulting tryptic peptides were lyophilized to dryness prior to offline fractionation (detailed below).

### Liver, Kidney, and Lung Tissue Homogenization

Human (JGH) and mouse (Silantes) fresh-frozen liver, kidney, and lung samples were cryo-homogenized using a prechilled (liquid nitrogen) mortar-and-hammer pestle. Homogenates were transferred to protein low-binding Eppendorf™ tubes containing protein extraction buffer (5% SDS, 100 mM TRIS pH 8.5, 10 mM TCEP) and subjected to probe-based ultrasonication (Fisherbrand™ Thermo Sonic Dismembrator) and heating to 95° C. for 10 minutes. Liver lysates were clarified by centrifugation (21000×g, 5 minutes) and approximately 5% of the sample was reserved for protein concentration determination by bicinchonic acid assay

### Liver, Kidney, and Lung SysQuan

Light human and heavy mouse tissue lysates (liver, kidney, or lung) were mixed 1:1 to generate a 100-μg human/mouse protein mix. Disulfide bonds were reduced in 10 mM dithiothreitol (DTT) for 30 minutes at 60° C. followed by alkylation in 50 mM IAA for 45 minutes at room temperature in the dark. Proteolytic digestion was performed using single-pot solid-phase-enhanced sample preparation (SP3) as described in Hughes, C.S., et al. Single-pot, solid-phase-enhanced sample preparation for proteomics experiments. Nat Protoc 14, 68-85 (2019). Prior to SP3, two types of carboxylate-modified Sera-Mag™ Speed beads (GE Life Sciences) were combined 1:1 (v/v), rinsed, and reconstituted in water at a concentration of 20μg solids/μL. Ten microliters of the prepared bead mix were added to the lysate and samples were adjusted to pH 7.0 using HEPES buffer. To promote proteins binding to the beads, acetonitrile (ACN) was added to a final concentration of 70% (v/v) and samples were incubated at room temperature on a tube rotator for 18 minutes. Subsequently, beads were immobilized on a magnetic rack for 1 minute. The supernatant was discarded, and the pellet was rinsed twice with 200 μL of 70% ethanol and once with 200 μL of 100% ACN while on the magnetic rack. The rinsed beads were resuspended in 115 μL of 50 mM HEPES buffer (pH 8.0) supplemented with trypsin (Promega™) at an enzyme-to-protein ratio of 1:25 (w/w) and incubated for 16 hours at 37° C. Peptide concentration was determined using Pierce™ Quantitative Fluorometric Peptide Assay (Thermo Fisher Scientific™).

Offline high pH reversed-phase fractionation. An Agilent™ 1100 series LC system with UV detector (214 nm) and a 1 mm×100 mm Gemini™ C18, 5 μm column (Phenomenex™, Torrance, CA) were used reversed-phase separation fractionation at pH 10. Both eluents (A) water and (B, 1:9 water: ACN) contained 20 mM ammonium formate pH 10. A linear gradient from 0.1-34% eluent B in 100 min was delivered at flow rate of 150 μL/min. 65 μg of the liver digest was injected and eighty 1-minute fractions were collected (between 10 and 90 minutes) and concatenated into 40 to provide optimal separation orthogonality. These fractions were lyophilized and resuspended in 0.1% formic acid (FA).

LC-MS/MS. Per fraction, 1 μg of total peptide was analyzed on an Orbitrap Exploris™ 480 (Thermo Fisher Scientific™, Bremen, Germany) coupled to an Easy-nLC™ 1000 (Thermo Fisher Scientific™) equipped with a C18 (Luna™ C18(2), 3 μm particle size (Phenomenex™, Torrance, CA)) column packed in-house in PicoFrit™ (100 μm×30 cm) capillaries (New Objective, Woburn, MA). Peptides were separated using a binary gradient with (A) 0.1% FA and (B) 0.1% (v/v) FA in 80% ACN (LC-MS grade), ramping from 0-5% B over 3 minutes, 5-7% B over 2 minutes, 7-25% B over 84 minutes, 25-60 % B over 15 minutes, 60-95% B over 1 minute at a flow rate of 300 nL/min. The Orbitrap Exploris™ 480 instrument was operated in data-dependent acquisition mode. Spray voltage was set to 2.4 kV, funnel radio frequency (RF) level at 40, and the heated capillary at 275° C. Survey scans covering the m/z 380-1500 were acquired at a resolution of 90,000 (at m/z 200), with a maximum ion injection time of 50 ms, and a normalized automatic gain control (AGC) target of 300%. Using the data-dependent acquisition (DDA) mode, selected ions were isolated with a width of m/z 1.6, a target automatic gain control (AGC) of 2e4 and maximum injection time set to auto. After fragmentation with a normalized collision energy of 30%, MS/MS spectra were acquired at a resolution of 30,000. Dynamic exclusion of previously selected ions was enabled for 30 seconds, charge state filtering was limited to 2-6, peptide match was set to preferred, and isotope exclusion was on.

### Liver, Kidney, and Lung SysQuan

The remaining tissue samples were reduced and alkylated with 10 mM TCEP and 40 mM IAA, respectively, and the equivalent of 200 μg of tissue lysate was digested with trypsin (Sigma-Aldrich™) at a 1:10 enzyme: substrate ratio at 37° C. for 16 hours using S-TRAP™ micro cartridges (ProtiFi™ LLC). Proteolysis was stopped with formic acid (0.5% v/v) and resulting tryptic peptides were lyophilized to dryness prior to offline fractionation (100 μg). Because protein yields were similar, the tissue lysates were combined volumetrically prior to offline high pH fractionation and subsequent LC-MS/MS analysis.

Offline high pH reversed-phase fractionation. To further increase the depth of coverage, we performed offline high-pH fractionation using an Agilent™ 1290 fraction collection system. Peptides were separated using a Waters™ XBridge™ peptide C18 column (2.1×150 mm, 2.1 μm particle) and peptides were separated using a binary gradient of (A) 10 mM ammonium formate (pH 10) and (B) acetonitrile (ACN) from 0% to 80% B over 48 minutes at flow rate of 400 μL/min with fractions collected every 30 seconds. Fractions from every 48^{th} sample (i.e., 1+49, 2+50, 3+51, etc.) were pooled and vacuum concentrated.

LC-MS/MS. Fractions from offline high-pH separation were reconstituted in 0.1% formic acid and analyzed by DDA-PASEF™ using an EvoSep™ One LC system (30SPD, EV1137 column) coupled to a Bruker™ timsTOF (trapped ion mobility time-of-flight) HT mass spectrometer using the DDA-PASEF™ standard method with a 1.1 s cycle time).

### Data Processing and Analysis

For each individual tissue sample set (plasma, liver, kidney, and lung) all raw data were searched using MSFragger (PMID: 28394336) embedded within the Fragpipe (v 21.1) interface (https://fragpipe.nesvilab.org/). Data were searched against the canonical human proteome downloaded from UniProtKB (UP000005640, downloaded May 2024 containing 20,467 protein sequences). The FASTA file was supplemented with reverse decoy sequences and common contaminants using Fragpipe. Searches were performed using default settings with labeled Lys (K+6. 020129 Da) set as a variable modification and including precursor quantitation based on SILAC with the following label types: light (K+0) and heavy (K+6.020129). Peptide and protein false discovery rates were set to ≤1%. Further processing and figure generation was performed in R using standard analysis and plotting functions.

### Example 1: Quantifying Liver, Kidney, Lung and Plasma Proteome by SysQuan

To evaluate the full potential of leveraging the proteomic proximity of mice and humans to use SysQuan as proteome-wide internal standards in the future, the inventors performed in-depth proteomics profiling of pooled human mouse liver, kidney, and lung tissue and plasma based on pre-fractionation using high pH reversed phase chromatography (see FIG. 1). The remarkable jump in LC-MS performance enabled by a recently introduced new type of hybrid mass spectrometer (Orbitrap™ ASTRAL™, Thermo Fisher Scientific™) that allows analyzing 5000-6000 proteins in less than 5 minutes, makes it reasonable to expect that a similar depth as achieved in the analysis will be feasible in 100×less instrument time in just a few years, i.e., unlocking SysQuan-based quantitation of 7000 liver proteins in 20 min.

Corresponding mouse and human tissues were cryogenically homogenized, proteins extracted, and heavy mouse and light human protein extracts were mixed 1:1 (wt/wt), followed reduction and alkylation and proteolytic digestion with trypsin. The mouse/human peptide mix was then fractionated using high pH reversed phase chromatography. This workflow was performed independently with slight deviations at two different labs where individual fractions were analyzed by DDA on an Orbitrap™ Exploris™ 480 (40 fractions) or DDA-PASEF on a timsTOF HT (48 fraction). Both datasets were processed with identical workflows using the Fragpipe software suite and SILAC settings.

### The SysQuan-Quantifable Proteome: Liver

In total 59,734 and 115,762 peptide-spectrum matches (PSM) were identified in the Exploris and timsTOF datasets, respectively, corresponding to 50,494 and 76,049 unique human peptide sequences, and 8323 and 8035 proteins. In the Exploris data, approximately 50% of the unique peptides, i.e. 23,727, had light/heavy pairs corresponding to 5,990 human proteins, and 19,004 non-modified mouse/human peptide pairs corresponded to 5,560 unique proteins.

In the timsTOF data, ⅔ of the unique peptides, i.e. 48,760, had light/heavy pairs, covering 6,987 unique human proteins, and 35,226 non-modified mouse/human peptide pairs corresponded to 6,305 unique proteins.

### The SysQuan-Quantifable Proteome: Kidney

Data generated from LC-MS/MS of the mixtures of unlabelled human kidney sample with the isotopically labelled murine kidney sample were analyzed with the SysQuan database search engine application integration (https://sysquan.com/). A total al 12650 unique proteins were identified. Of these, about 78% (or 9834 proteins) had light/heavy peptide doublets and were therefore quantifiable.

### The SysQuan-Quantifable Proteome: Lung

Data generated from LC-MS/MS of the mixtures of unlabelled human lung sample with the isotopically labelled murine lung sample were analyzed with the SysQuan database search engine application integration (https://sysquan.com/). A total al 12712 unique proteins were identified. Of these, about 76% (or 9620 proteins) had light/heavy peptide doublets and were therefore quantifiable.

### The SysQuan-Quantifable Proteome: Plasma

Mouse and human plasma were depleted of the top 14 most abundant proteins and pooled 1:1 and processed as described for liver. A total of 48 high pH reversed phase fractions were analyzed by PASEF-DDA on a timsTOF HT. Fragpipe with SILAC settings yielded 23,501 PSM corresponding to 15,302 unique peptides and 2,279 unique proteins. More than ⅓, i.e., 5,769 peptides corresponding to 1,431 proteins had light/heavy pairs, and 4,020 non-modified mouse/human peptide pairs corresponded to 1,209 proteins.

### Example 2: Comparison to Reference Proteomes

To contextualize the depth of the SysQuan-quantifiable proteomes, the inventors compared their data with the current reference proteomes from the proteomics community. The latest build of the Human Plasma Proteome Project (HPPP) of the Human Proteome Organization (https://hupo.org/Human-Plasma-Proteome-Project-(HPPP)), combines results from 313 experiments and comprises 4,608 human plasma proteins. The inventors identified 2,279 proteins, 1,431 of which can be quantified using SysQuan. Thus, with a single workflow and proteolytic enzyme, SysQuan enables quantitation of approximately 31% of the known plasma proteome. Diversifying the proteomics workflow to better reflect the 313 experiments in HPPP may allow quantifying up to ⅔ of the plasma proteome in accordance with in silico predictions and the inventors'own plasma data (quantifiable/identified). Integration of SysQuan into recent workflows that considerably increased the depth of plasma proteomics may help reach this goal.

The human liver atlas contains 10,528 unique proteins, identified through a major effort including the use of diverse immortalized liver cell lines, primary cells, and human biopsies, for a total of 34 different sample types. In each experiment, the inventors identified between 9,400 and 9,730 proteins. Here, 96% of the known human liver proteome were identified, and the data indicates that 70% of this known liver proteome is quantifiable using SysQuan. This number aligns well with the in silico predictions.

### Example 3: Labeling Efficiency

To determine labeling efficiency, the inventors analyzed pure SILAC mouse liver by LC-MS/MS and analyzed the data as described for the mouse/human mixes, using SILAC ratios to determine labeling efficiencies for all identified mouse peptides. More than 66% of all identified mouse liver peptide pairs contained more than 90% SILAC peptide, i.e., >90% labeled. Only 10% of all proteins had labeling levels of below 50%. An enrichment analysis localized this set of proteins to the cytoskeleton, plasma membrane, and intracellular vesicle with functions related to protein binding. These are proteins with slow turnover, which explains their lower labeling rates. The inventors were also able to determine that 90% of all proteins have at least one peptide with 100% labelling efficiency. Determining the labeling efficiency on the peptide and protein level allows advanced selection of the most suitable surrogate (i.e., proteotypic) peptides for quantification. Typically, peptides with a labelling efficiency of 95% or higher are suitable for quantification.

### Example 4: Translating SysQuan to Targeted Proteomics

The fractionation-based deep proteomic profiling of plasma and liver amounted to nearly 2 days of LC-MS instrument time, each. The intent was to assess the utility of SysQuan to function as internal standard for quantitative proteomics. As mentioned above, the inventors believe that the continuous advance of MS-based proteomics technologies will make quantification by SysQuan achievable at a fraction of the current measurement time in the future.

A technology that can unlock the extended depth of the proteome is targeted proteomics using multiple reaction monitoring (MRM) and parallel reaction monitoring (PRM), which involves quantification of predefined sets of peptides using high resolution mass spectrometry. Targeted proteomics has been shown to extend the accessible dynamic range, enables access to low abundance proteins and peptides that might be lost during non-targeted analyses, while concurrently providing improved quantitative precision. Recent studies have shown that targeted MS can be used to target certain proteins in urine and plasma, covering a dynamic range of orders of magnitude. Recent technological advancements enable reducing the LC-MS instrument time for multiplexed MRM of plasma proteins while maintaining depth and quantitative precision. Based on these results, the inventors believe that already with the presently available technology, targeted proteomics can be used to quantify thousands of predefined proteins in a short time frame (i.e., targeting light and heavy forms of a given peptide). While this is not comparable to the proteome depth of non-targeted approaches, targeted MS provide improved precision and unlocks access to a wider dynamic range, such that it particularly enables quantitation of very low-abundance targets that may be hard to access for (precise) quantitation otherwise. This is particularly important as in many studies, precise measurement of a smaller but defined set of proteins across a large number of samples and without missing values is preferrable over proteome-wide analysis with less precision and more incomplete data.

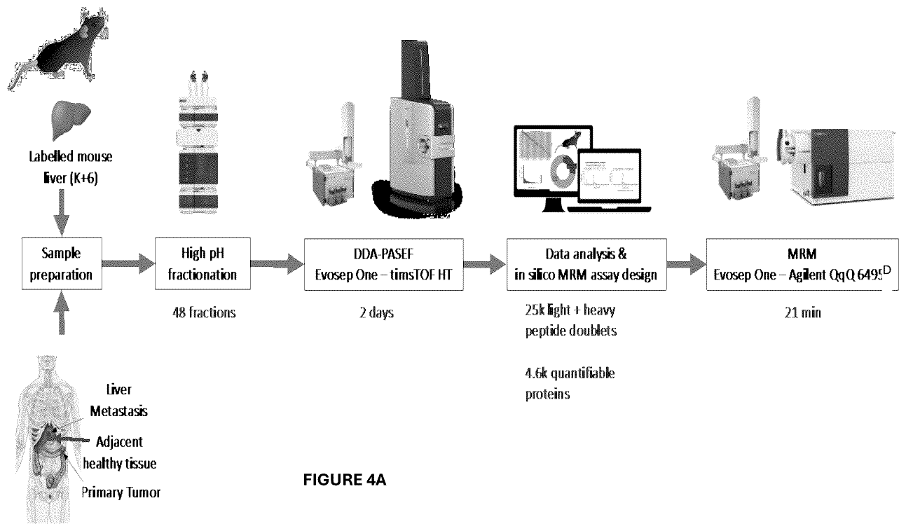

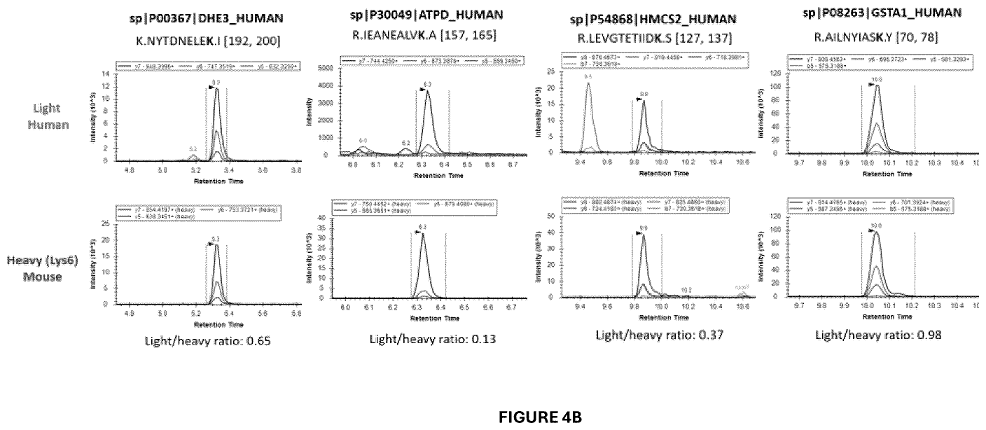

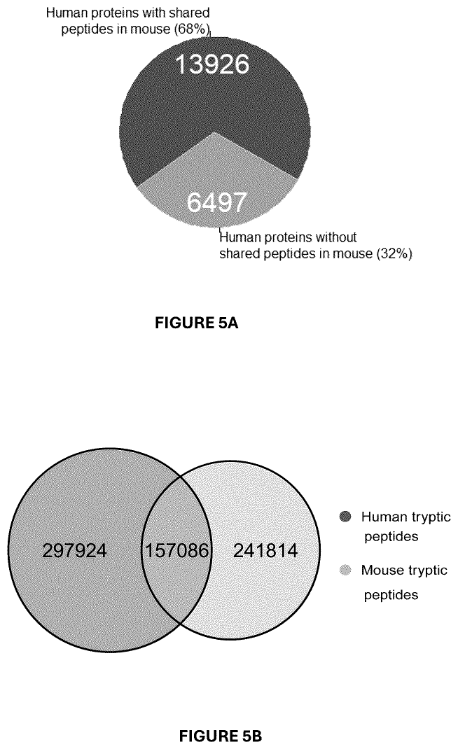

The inventors, therefore, developed and tested a pipeline to transfer SysQuan targets from their deep proteomics discovery data to a targeted quantitative proteomics workflow using an Agilent 6495C triple quadrupole mass spectrometer (see FIG. 4A). Information was used from the discovery data, notably acquired under a completely different LC-MS platform, to successfully predict for selected targets the best MRM transitions as well as retention times, in order to use scheduled MRM. The inventors chose a total of 14 proteins with important roles in metabolism.

While the invention has been described in connection with specific embodiments thereof, it will be understood that the scope of the claims should not be limited by the preferred embodiments set forth in the examples, but should be given the broadest interpretation consistent with the description as a whole.

1. A kit for a high-throughput proteomics method of identifying and/or quantifying proteins from a biological sample obtained from a subject, the kit comprising (i) one or more internal standards comprising isotope labelled murine proteins obtained from a biological sample from a mouse fed isotopes; and (ii) instructions for co-digesting proteins in the biological sample from a mouse fed isotopes and a corresponding biological sample obtained from the subject, and for identifying un-labelled and labelled corresponding doublets of peptides in a mass spectrometry analysis from a lysate mixture from the co-digestion to quantify the proteins from the biological sample from the subject.

2. The kit of claim 1, the biological sample from the mouse is a dried biological sample.

3. The kit of claim 2, wherein the dried biological sample is a lyophilized sample.

4. The kit of claim 3, wherein the lyophilized sample is in a powdered form.

5. The kit of claim 1, wherein the kit is for quantifying proteins in a sex-specific analysis of the biological sample obtained from a female or male subject, the kit comprising a dried biological sample comprising isotope labelled proteins from one of a respective female mouse or male mouse.

6. The kit of claim 2, wherein the dried biological sample of the mouse is from plasma, kidney, lung, heart, liver.

7. The kit of claim 1, wherein the instructions comprise steps for quantifying the proteins using a high-throughput method for identifying and/or quantifying proteins from a biological sample obtained from a subject, the method comprising:
(i) providing the biological sample from the subject, wherein the biological sample is from an organ, fluid, or tissue;
(ii) homogenizing the biological sample from the subject and extracting proteins therefrom;
(iii) co-digesting proteins in the biological sample obtained from the subject with a corresponding biological sample obtained from a mouse comprising murine proteins, which murine proteins are isotope labelled,
the co-digesting producing a lysate mixture comprising a mixture of unlabelled peptides from the biological sample and corresponding isotope labelled murine peptides;
(iv) analyzing the peptides in the lysate mixture by mass spectrometry, wherein the isotope labelled peptides in the murine sample exhibit a measurable mass difference relative to unlabelled peptides from the biological sample from the subject;
(v) identifying unlabelled and labelled corresponding doublets of peptides from the lysate mixture; and
(vi) quantifying the relative abundance of the peptides in the biological sample by comparing an intensity of mass spectrometry signals of the isotope labelled peptides to the unlabelled peptides of the doublets.

8. The kit of claim 1, wherein the instructions include software for quantifying the proteins.

9. The kit of claim 1, wherein the instructions comprise standard operating procedures, reagents, consumables, one or more known protein concentrations from a database and/or data analysis software or reference to said software.

10. A lyophilized internal standard comprising dried isotope labelled murine proteins.

11. The lyophilized internal standard of claim 10, wherein the isotope labelled murine proteins are from a female mouse.

12. The lyophilized internal standard of claim 10, wherein the isotope labelled murine proteins are from a male mouse.

13. A high-throughput method for identifying and/or quantifying proteins from a biological sample obtained from a subject, the method comprising:
(i) providing the biological sample from the subject, wherein the biological sample is from an organ, fluid, or tissue;
(ii) homogenizing the biological sample from the subject and extracting proteins therefrom;
(iii) co-digesting proteins in the biological sample obtained from the subject with a corresponding biological sample obtained from a mouse comprising murine proteins, which murine proteins are isotope labelled,
the co-digesting producing a lysate mixture comprising a mixture of un-labelled peptides from the biological sample and corresponding isotope labelled murine peptides;
(iv) analyzing the peptides in the lysate mixture by mass spectrometry, wherein the isotope labelled peptides in the murine sample exhibit a measurable mass difference relative to unlabelled peptides from the biological sample from the subject;
(v) identifying unlabelled and labelled corresponding doublets of peptides from the lysate mixture; and
(vi) quantifying the relative abundance of the peptides in the biological sample by comparing an intensity of mass spectrometry signals of the isotope labelled peptides to the unlabelled peptides of the doublets.

14. The method of claim 13, wherein the biological sample obtained from the subject is the fluid.

15. The method of claim 13, wherein the fluid is plasma and wherein the proteins extracted in step (ii) are plasma proteins.

16. The method of claim 12, wherein the biological sample obtained from the subject is from an organ.

17. The method of claim 15, wherein the organ is selected from one of kidney, lung, heart and liver.

18. The method of claim 15, wherein the organ is liver.

19. A method for absolute quantification of a protein comprising:
(i) preparing a chemically synthesized unlabeled peptide (NAT) based on a peptide quantified in the method of claim 13;
(ii) combining the NAT with one or more isotope-labelled peptides from a mouse to produce a NAT/mouse labelled peptide mixture; and
(iii) analyzing the one or more peptides in the peptide mixture by mass spectrometry, comprising identifying unlabelled and labelled corresponding doublets of the one or more peptides from the peptide mixture; and quantifying the relative abundance of the one or more peptides in the biological sample by comparing an intensity of mass spectrometry signals of the isotope labelled peptides to the unlabelled peptides of the doublets.

20. The method of claim 19, wherein the one or more isotope labelled peptides of step (ii) are stable isotope labelled standard peptides derived from SILAC (SILAC SIS) peptides.
